(19) United States
(12) Patent Application Publication
(10) Pub. No.: US 2006/0034902 A1
(43) Pub. Date: Feb. 16, 2006

(54) Microprojection apparatus and system with low infection potential

(76) Inventors: Michel J.N. Cormier, Mountain View, CA (US); Peter Daddona, Menlo Park, CA (US); Rolfe Anderson, Saratoga, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/201,617

(22) Filed: Aug. 10, 2005

Related U.S. Application Data
(60) Provisional application No. 60/600,638, filed on Aug. 10, 2004.

(51) Int. Cl. A61F 13/02; A61L 15/16

(52) U.S. Cl. 424/448; 604/500

(57) ABSTRACT

A transdermal agent delivery apparatus and system having a low infection potential comprising a delivery system having a microprojection member (or system) that includes a plurality of microprojections (or array thereof) that are adapted to pierce through the stratum corneum into the underlying epidermis layer, or epidermis and dermis layers. In one embodiment, the microprojection member includes a biocompatible coating having at least one biologically active agent and at least one antimicrobial agent disposed therein. In another embodiment, the microprojection member includes a hydrogel formulation having at least one biologically active agent and at least one antimicrobial agent. In yet another embodiment, the microprojection member includes a hydrogel formulation having at least one antimicrobial agent and a solid film having at least one biologically active agent.

### CROSS REFERENCE TO RELATED APPLICATIONS

This application claims the benefit of U.S. Provisional Application Ser. No. 60/600,638, filed Aug. 10, 2004.

### FIELD OF THE PRESENT INVENTION

The present invention relates generally to transdermal agent delivery apparatus and systems. More particularly, the invention relates to a transdermal agent delivery apparatus and system having a low infection potential.

### BACKGROUND OF THE INVENTION

Active agents (or drug) are most conventionally administered either orally or by injection. Unfortunately, many active agent are completely ineffective or have radically reduced efficacy when orally administered, since they either are not absorbed or are adversely affected before entering the bloodstream and thus do not possess the desired activity. On the other hand, the direct injection of the agent into the bloodstream, while assuring no modification of the agent during administration, is a difficult, inconvenient, painful and uncomfortable procedure which sometimes results in poor patient compliance.

Hence, in principle, transdermal delivery provides for a method of administering active agents that would otherwise need to be delivered via hypodermic injection or intravenous infusion. The word “transdermal”, as used herein, is generic term that refers to delivery of an active agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery includes intracutaneous, intradermal and intraepidermal delivery via passive diffusion as well as delivery based upon external energy sources, such as electricity (e.g., iontophoresis) and ultrasound (e.g., phonophoresis).

Passive transdermal agent delivery systems, which are more common, typically include a reservoir that contains a high concentration of an active agent. The reservoir is adapted to contact the skin, which enables the agent to diffuse through the skin and into the body tissues or bloodstream of a patient.

As is well known in the art, the transdermal agent flux is dependent upon the condition of the skin, the size and physical/chemical properties of the agent molecule, and the concentration gradient across the skin. Because of the low permeability of the skin to many active agents, transdermal delivery has had limited applications. This low permeability is attributed primarily to the stratum corneum, the outermost skin layer (see FIG. 1). The stratum corneum generally consists of flat, dead cells filled with keratin fibers (i.e., keratinocytes) surrounded by lipid bilayers. This highly-ordered structure of the lipid bilayers confers a relatively impermeable character to the stratum corneum.

One common method of increasing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal protein flux has been limited, at least for the larger proteins, due to their size.

There also have been many techniques and apparatus developed to mechanically penetrate or disrupt the outermost skin layers thereby creating pathways into the skin in order to enhance the amount of agent being transdermally delivered. Illustrative is the drug delivery apparatus disclosed in U.S. Pat. No. 3,964,482.

Other systems and apparatus that employ tiny skin piercing elements (i.e., microprojections) to enhance transdermal agent delivery are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The noted systems and apparatus typically include a reservoir for holding the agent and also a delivery system to transfer the agent from the reservoir through the stratum corneum, such as by hollow tines of the device itself. One example of such a device is disclosed in WO 93/17754, which has a liquid agent reservoir.

As disclosed in U.S. patent application Ser. No. 10/045,842, which is fully incorporated by reference herein, it is also possible to have the active agent that is to be delivered coated on the microprojections instead of contained in a physical reservoir. This eliminates the necessity of a separate physical reservoir and developing an agent formulation or composition specifically for the reservoir. Illustrative are the Macroflux® apparatus and systems disclosed in U.S. application Ser. Nos. 08/988,292; 09/950,436; 09/976,762; 09/976,798; 10/045,842; 10/127,108; 10/327,330; 10/674,626; 10/608,304.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the outermost skin layer (i.e., stratum corneum) for enhancing transdermal agent delivery therethrough.

As is well known in the art, the stratum corneum constitutes a natural barrier against potential local infection from residence microbial fiora. Breaching of the stratum corneum thus potentially opens the door to local skin infection.

As is also well known in the art, the risk of infection is dependent on the number and nature of the microorganism introduced into the host's body, the immune response of the host, the occlusion time and the composition of the occluding medium.

It is also probable that the risk of infection will increase with the depth of penetration of the microprojections into the skin and the number of microprojections penetrating the skin,

It is also probable that the risk of infection will increase with increased wearing time, especially in the case where the formulation can support microbial growth, like with hydrated agent-containing reservoir.

Although the risks of infections through the use of the noted microprojection apparatus, particularly, the Macroflux® apparatus, is minimal by virtue of several factors (e.g., short residence time, coated Macroflux® systems do not sustain bacterial growth), it would be desirable to provide a microprojection apparatus and system with a low infection potential.

It is therefore an object of the present invention to provide a transdermal agent delivery apparatus and system having a low infection potential that provides intracutaneous delivery of a biologically active agent to a subject.

It is another object of the present invention to provide a transdermal agent delivery apparatus and system that prevents microbial growth during manufacturing.

It is another object of the present invention to provide a transdermal agent delivery apparatus and system that prevents microbial growth during storage.

It is another object of the present invention to provide a transdermal agent delivery apparatus and system that substantially reduces or eliminates microbial growth following application of the apparatus to the skin of a subject.

It is yet another object of the invention to provide a biologically active agent incorporating at least one antimicrobial agent formulation for intracutaneous delivery to a patient.

### SUMMARY OF THE INVENTION

In accordance with the above objects and those that will be mentioned and will become apparent below, the transdermal agent delivery apparatus and system having a low infection potential in accordance with this invention includes a microprojection member (or system) that includes a plurality of microprojections (or array thereof) that are adapted to pierce through the stratum corneum into the underlying epidermis layer, or epidermis and dermis layers. In one embodiment, the microprojection member includes a biocompatible coating having at least one biologically active agent and at least one antimicrobial agent disposed therein. In another embodiment, the microprojection member includes a hydrogel formulation having at least one biologically active agent and at least one antimicrobial agent. In yet another embodiment, the microprojection member includes a hydrogel formulation having at least one antimicrobial agent and a solid film having at least one biologically active agent.

In one embodiment of the invention, the microprojection member has a microprojection density of at least approximately 10 microprojections/cm², more preferably, in the range of at least approximately 200-2000 microprojections/cm².

In one embodiment, the microprojection member is constructed out of stainless steel, titanium, nickel titanium alloys, or similar biocompatible materials, such as polymeric materials.

In another embodiment, the microprojection member is constructed out of a non-conductive material, such as a polymer. Alternatively, the microprojection member can be coated with a non-conductive material, such as Parylene®, or a hydrophobic material, such as Teflon®, silicon or other low energy material.

Preferably, the antimicrobial agent is selected from the group consisting of 2-bromo-2-nitropropane-1,3-diol, 5-bromo-5-nitro-1,3-dioxane, 7-ethyl bicyclooxazolidine, benzalkonium chloride, benzethonium chloride, benzoic acid, benzyl alcohol, boric acid, bronopol, cetylpyridinium chloride, chlorhexidine digluconate, chloroacetamide, chlorobutanol, chloromethyl isothiazolinone and methyl isothiazoline, dimethoxane, dimethyl oxazolidine, dimethyl hydroxymethyl pyrazole, chloroxylenol, dehydroacetic acid, diazolidinyl urea, dichlorobenzyl alcohol, DMDM hydantoin, ethyl alcohol, formaldehyde, glutaraldehyde, hexachlorophene, hexetidine, hexamethylenetramine, imidazolidinyl urea, iodopropynyl butylcarbamate, isothiazolinones, methenammonium chloride, methyldibromo glutaronitrile, MDM hydantoin, ortho phenylphenol, p-chloro-m-cresol, parabens (butylparaben, ethylparaben, methylparaben), phenethyl alcohol, phenoxyethanol, piroctane olamine, polyaminopropyl biguanide, polymethoxy bicyclic oxazolidine, polyoxymethylene, polyquaternium-42, potassium benzoate, potassium sorbate, propionic acid, quaternium-15, salicylic acid, selenium disulfide, sodium borate, sodium iodate, sodium hydroxymethylglycinate, sodium propionate, sodium pyrithione, sorbic acid, thimerosal, triclosan, triclocarban, undecylenic acid, zinc phenosulfonate, and zinc pyrithione

In a preferred embodiment of the invention, the biologically active agent is selected from the group consisting of small molecular weight compounds, polypeptides, proteins, oligonucleotides, nucleic acids and polysaccharides.

In one embodiment of the invention, the biologically active agent is selected from the group consisting of leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N-[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, VEGF, BNP, ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormones (PTH), parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, analgesics, such as fentanyl, remifentanyl, sufentanyl, alfentanyl, lofentanyl, carfentanyl, and analogues and mixtures thereof.

In another embodiment of the invention, the biologically active agent comprises a vaccine. The vaccine can comprise viruses and bacteria, protein-based vaccines, polysaccharide-based vaccine, and nucleic acid-based vaccines.

Suitable antigenic agents include, without limitation, antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linke to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diphtheriae, group A streptococcus, legionella pneumophila, neisseria meningitdis, pseudomonas aeruginosa, streptococcus pneumoniae, treponema pallidum, and vibrio cholerae, and mixtures thereof.

Additional commercially available vaccines, which contain antigenic agents, include, without limitation, flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: β-glucan; cholera toxin B subunit; CRL1005: ABA block polymer with mean values of x=8 and y=205; gamma inulin: linear (unbranched) β-D (2→1) polyfructofuranoxyl-α-D-glucose; Gerbu adjuvant: N-acetylglucosamine-(β1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo [4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂0 (MTP); Murametide: Nac-Mur-L-Ala-D-Gln-OCH₃; Pleuran: β-glucan; QS-21; S-28463: 4-amino-a,a-dimethyl-1H-imidazo[4,5-c]quinoline-1-ethanol; sclavo peptide: VQGEESNDK∘HCl (IL-β 163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

The coating formulations applied to the microprojection member to form solid biocompatible coatings can comprise aqueous and non-aqueous formulations. In a preferred embodiment, the coating formulations include at least one antimicrobial agent and at least one biologically active agent, which can be dissolved within a biocompatible carrier or suspended within the carrier

Preferably, the antimicrobial agent comprises in the range of approximately 0.005-5.0 wt. % of the coating formulation.

In one embodiment of the invention, wherein ethanol is employed as a preservative, the antimicrobial agent comprises up to approximately 20 wt. % of the coating formulation.

Preferably, the biologically active agent comprises in the range of approximately 0.1-30 wt. % of the coating formulation.

In one embodiment of the invention, the coating formulation includes at least one buffer. Examples of such buffers include ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine or mixtures thereof.

In one embodiment of the invention, the coating formulation includes at least one surfactant, which can be zwitterionic, amphoteric, cationic, anionic, or nonionic. Examples of such surfactants include, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives, such as sorbitan laurate, and alkoxylated alcohols, such as laureth-4.

In one embodiment of the invention, the concentration of the surfactant is in the range of approximately 0.001-2.0 wt. % of the coating formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxy-ethylcellulose (EHEC), as well as pluronics.

In one embodiment of the invention, the concentration of the polymer presenting amphiphilic properties in the coating formulation is preferably in the range of approximately 0.01-20 wt. %, more preferably, in the range of approximately 0.03-10 wt. % of the coating formulation.

In another embodiment, the coating formulation includes a hydrophilic polymer selected from the following group: hyroxyethyl starch, dextran, poly(vinyl alcohol), poly(ethylene oxide), poly(2-hydroxyethylmethacrylate), poly(n-vinyl pyrolidone), polyethylene glycol and mixtures thereof.

In a preferred embodiment, the concentration of the hydrophilic polymer in the coating formulation is in the range of approximately 0.01-20 wt. %, more preferably, in the range of approximately 0.3-10 wt. %.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier in the coating formulation is in the range of approximately 2-70 wt. %, more preferably, in the range of approximately 5-50 wt. % of the coating formulation.

In another embodiment, the coating formulation includes a stabilizing agent, which can comprise, without limitation, a non-reducing sugar, a polysaccharide or a reducing sugar or a DNase inhibitor.

Suitable non-reducing sugars include, for example, sucrose, trehalose, stachyose, or raffinose.

Suitable polysaccharides include, for example, dextran, soluble starch, dextrin, and inulin.

Suitable reducing sugars include, for example, monosaccharides, such as apiose, arabinose, lyxose, ribose, xylose, digitoxose, fucose, quercitol, quinovose, rhannose, allose, altrose, fructose, galactose, glucose, gulose, hamamelose, idose, mannose, tagatose, and the like; and disaccharides, such as primeverose, vicianose, rutinose, scillabiose, cellobiose, gentiobiose, lactose, lactulose, maltose, melibiose, sophorose, and turanose, and the like.

Suitable DNase inhibitors include, for example, both extracellular and intracellular DNase inhibitors. Preferred extracellular DNase inhibitors include, for example, aurintricarboxylic acid (ATA); EDTA; EGTA; and propamidine. Preferred intracellular DNases inhibitors include, for example, DMI-2, which is a polyketice metabolite of Streptomyces sp. Strain 560. In preferred embodiments of the invention, the compositions and solid coatings comprise from about 1% to about 20% by total dry weight of the DNase inhibitor.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating formulation.

In another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the coating formulation includes a solubilising/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin.

The concentration of the solubilising/complexing agent, if employed, is preferably in the range of approximately 1 wt. % to 20 wt. % of the coating formulation.

In another embodiment of the invention, the coating formulation includes at least one non-aqueous solvent, such as ethanol, isopropanol, methanol, propanol, butanol, pentanol, acetone, ethyl ether, benzene, amylene hydrate, methyl isobutyl ketone, propylene glycol, glycerol, and polyethylene glycols. Preferably, the solvent is present in the coating formulation in the range of approximately 5 wt. % to 99 wt. % of the coating formulation.

Preferably, the coating formulations have a viscosity less than approximately 500 centipoise and greater than 3 centipoise.

In one embodiment of the invention, the thickness of the biocompatible coating is less than 25 microns, more preferably, less than 10 microns, as measured from the microprojection surface.

The hydrogel formulations of the invention preferably comprise aqueous formulations. In one embodiment of the invention, the hydrogel formulations include at least one antimicrobial agent and at least one biologically active agent, which can be dissolved or suspended in the hydrogel formulation.

In a preferred embodiment of the invention, the microprojection member includes a gel pack that is adapted to receive the hydrogel formulation.

Preferably, the antimicrobial agent comprises in the range of approximately 0.005-5 wt. % of the hydrogel formulation.

In one embodiment of the invention, wherein ethanol is employed as a preservative, the antimicrobial agent comprises up to 20 wt. % of the hydrogel formulation.

Preferably, the biologically active agent comprises in the range of approximately 0.1-30 wt. % of the hydrogel formulation.

In one embodiment of the invention, the hydrogel formulation includes at least one of the aforementioned buffers.

The hydrogel formulation of the invention preferably comprise water-based hydrogels having macromolecular polymeric networks.

In a preferred embodiment of the invention, the polymer network comprises, without limitation, hyroxyethyl starch, dextran, hydroxyethylcellulose (HEC), hydroxypropylmethylcellulose (HPMC), hydroxypropycellulose (HPC), methylcellulose (MC), hydroxyethyl-methylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics.

The hydrogel formulation preferably includes at least one surfactant, which can be zwitterionic, amphoteric, cationic, anionic, or nonionic.

In one embodiment of the invention, the surfactant comprises sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, and alkoxylated alcohols such as laureth-4.

In another embodiment, the hydrogel formulation includes polymeric materials or polymers having amphiphilic properties, which can comprise, without limitation, cellulose derivatives, such as hydroxyethylcellulose (HEC), hydroxypropyl-methylcellulose (HPMC), hydroxypropycellulose (HPC), methylcellulose (MC), hydroxyethylmethylcellulose (HEMC), or ethylhydroxyethylcellulose (EHEC), as well as pluronics.

In a further embodiment of the invention, the hydrogel formulation includes a solubilising/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl-beta-Cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin. Most preferred are beta-cyclodextrin, hydroxypropyl-beta-Cyclodextrin, 2-hydroxypropyl-beta-Cyclodextrin and sulfobutylether7 beta-cyclodextrin.

In another embodiment of the invention, the hydrogel formulation includes at least one non-aqueous solvent, such as ethanol, isopropanol, acetone, propylene glycol, glycerol, and polyethylene glycols. Preferably, the solvent is present in the hydrogel formulation in the range of approximately 5 wt. % to 75 wt. % of the formulation.

In accordance with yet another embodiment of the invention, microprojection member includes top and bottom surfaces, a plurality of openings that extend through the microprojection member and a plurality of stratum corneum-piercing microprojections that project from the bottom surface of the microprojection member. The microprojection member further includes a hydrogel formulation and a solid agent-containing film. Preferably, the agent-containing film includes at least one biologically active agent, more preferably, the agent-containing film includes at least one antimicrobial agent and at least one biologically active agent.

In one embodiment, the solid film is disposed proximate the top surface of the microprojection member. In another embodiment, the solid film is disposed proximate the bottom surface of the microprojection member.

In a preferred embodiment, the hydrogel formulation contains at least one antimicrobial agent and is devoid of a biologically active agent.

In one embodiment, the solid film is made by casting a liquid formulation consisting of at least one antimicrobial agent, at least one biologically active agent, a polymeric material, such as hyroxyethyl starch, dextran, hydroxyethylcellulose (HEC), hydroxypropylmethylcellulose (HPMC), hydroxypropycellulose (HPC), methylcellulose (MC), hydroxyethylmethylcellulose (HEMC), ethylhydroxethylcellulose (EHEC), carboxymethylcellulose (CMC), poly(vinyl alcohol), poly(ethylene oxide), poly(2-hydroxyethymethacrylate), poly(n-vinyl pyrolidone), or pluronics, a plasticising agent, such as glycerol, propylene glycol, or polyethylene glycol, a surfactant, such as tween 20 or tween 80, and at least one volatile solvent, such as water, isopropanol, methanol, ethanol, or acetone.

In one embodiment, the liquid formulation used to produce the solid film comprises: 0.005-5 wt. % antimicrobial agent, 0.1-20 wt. % biologically active agent, 5-40 wt. % polymer, 5-40 wt. % plasticiser, 0-2 wt. % surfactant, and the balance comprising a volatile solvent.

In one embodiment of the invention, the liquid formulation used to produce the solid film includes at least one of the aforementioned buffers.

In another embodiment of the invention, the liquid formulation used to produce the solid film includes at least one of the aforementioned complexing/solubilising agents.

In a further embodiment of the invention, the liquid formulation used to produce the solid film includes at least one of the aforementioned vasoconstrictors.

In a further embodiment of the invention, the liquid formulation used to produce the solid film includes at least one of the aforementioned pathway patency modulators.

In accordance with one embodiment of the invention, the method for delivering an agent formulation of the invention includes the following steps: (i) providing a delivery system having a microprojection member, the microprojection member including a plurality of microprojections and a biocompatible coating having at least one biologically active agent and at least one antimicrobial agent disposed therein, (ii) applying the coated microprojection member to the patient's skin via an actuator, wherein the microprojections pierce the skin and the agents-containing coating is dissolved by body fluid and released into the skin.

The coated microprojection member is preferably left on the skin for a period lasting from 5 seconds to 24 hours. Following the desired wearing time, the microprojection member is removed from the skin.

In accordance with a further embodiment of the invention, the method for delivering an agent formulation of the invention includes the following steps: (i) providing a delivery system having a microprojection member and a gel pack including a hydrogel formulation having at least one biologically active agent and at least one antimicrobial agent, (ii) applying the microprojection member to the patient's skin via an actuator, wherein the microprojections pierce the stratum corneum and (iii) placing the gel pack on top of the applied microprojection member, wherein the hydrogel formulation migrates into and through the microslits in the stratum corneum produced by the microprojections.

The microprojection member-gel pack assembly is preferably left on the skin for a period lasting from 5 minutes to 7 days. Following the desired wearing time, the microprojection member is removed from the skin.

In a further aspect of the noted embodiment, the microprojection member includes an agent-containing biocompatible coating and wherein the antimicrobial agent is present in the hydrogel formulation and/or the biocompatible coating, the biologically active agent is contained in the biocompatible coating, and the hydrogel formulation is devoid of a biologically active agent and, hence, is merely a hydration mechanism.

In accordance with another embodiment of the invention, the method for delivering an agent formulation of the invention includes the following steps: (i) providing a delivery system having a microprojection member and a gel pack including a hydrogel formulation having at least one biologically active agent and at least one antimicrobial agent, (ii) applying the microprojection member to the patient's skin via an actuator, wherein the microprojections pierce the stratum corneum, (iii) removing the microprojection member from the patient's skin and (iv) placing the gel pack on top of the pretreated skin, wherein the hydrogel formulation migrates into and through the microslits in the stratum corneum produced by the microprojections.

The gel pack is preferably left on the skin for a period lasting from 5 minutes to 7 days. Following the desired wearing time, the gel pack is removed from the skin.

In a further embodiment of the invention, the method for delivering an agent formulation of the invention includes the following steps: (i) providing a delivery system having a microprojection member, a gel pack including a hydrogel formulation, and a solid film having at least one biologically active agent and at least one antimicrobial agent, and (ii) applying the microprojection member to the patient's skin via an actuator, wherein the microprojections pierce the stratum corneum, the hydrogel formulation hydrates and releases the agent formulation from the solid film and the agent formulation migrates into and through the microslits in the stratum corneum produced by the microprojections.

The microprojection member is preferably left on the skin for a period lasting from 5 seconds to 24 hours. Following the desired wearing time, the microprojection member is removed from the skin.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is an illustration of a human host's skin, illustrating the stratum corneum, epidermis and dermis layers;

FIG. 2 is a perspective view of a portion of one example of a microprojection member;

FIG. 3 is a perspective view of the microprojection member shown in FIG. 2 having a coating deposited on the microprojections, according to the invention;

FIG. 4 is a side sectional view of a microprojection member having an adhesive backing;

FIG. 5 is an exploded perspective view of one embodiment of a gel pack of a microprojection system;

FIG. 6 is an exploded perspective view of one embodiment of a microprojection member of a microprojection system;

FIG. 7 is a perspective view of one embodiment of a microprojection assembly comprising the gel pack shown in FIG. 5 and the microprojection member shown in FIG. 6;

FIG. 8 is a side sectional view of a retainer having a microprojection member disposed therein;

FIG. 9 is a perspective view of the retainer shown in FIG. 7;

FIG. 10 is an exploded perspective view of an applicator and retainer.

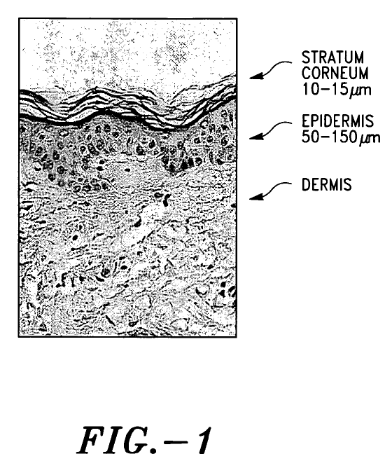

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an antimicrobial agent” includes two or more such agents; reference to “a microprojection” includes two or more such microprojections and the like.

### DEFINITIONS

The term “transdermal”, as used herein, means the delivery of an agent into and/or through the skin for local or systemic therapy. The term “transdermal” thus means and includes intracutaneous, intradermal and intraepidermal delivery of an active agent into and/or through the skin via passive diffusion as well as energy-based diffusional delivery, such as iontophoresis and phonophoresis.

The term “transdermal flux”, as used herein, means the rate of transdermal delivery.

The term “co-delivering”, as used herein, means that a supplemental agent is administered transdermally either before the antimicrobial agent and/or biologically active agent is delivered, before and during transdermal flux of the antimicrobial agent and/or biologically active agent, during transdermal flux of the antimicrobial agent and/or biologically active agent, during and after transdermal flux of the antimicrobial agent and/or biologically active agent, and/or after transdermal flux of the antimicrobial agent and/or biologically active agent. Additionally, two or more antimicrobial and/or biologically active agents may be formulated in the coatings and/or hydrogel formulations and/or solid films of the invention, resulting in co-delivery of the antimicrobial and/or biologically active agents.

The term “antimicrobial agent”, as used herein, includes, without limitation 2-bromo-2-nitropropane-1,3-diol, 5-bromo-5-nitro-1,3-dioxane, 7-ethyl bicyclooxazolidine, benzalkonium chloride, benzethonium chloride, benzoic acid, benzyl alcohol, boric acid, bronopol, cetylpyridinium chloride, chlorhexidine digluconate, chloroacetamide, chlorobutanol, chloromethyl isothiazolinone and methyl isothiazoline, dimethoxane, dimethyl oxazolidine, dimethyl hydroxymethyl pyrazole, chloroxylenol, dehydroacetic acid, diazolidinyl urea, dichlorobenzyl alcohol, DMDM hydantoin, ethyl alcohol, formaldehyde, glutaraldehyde, hexachlorophene, hexetidine, hexamethylenetramine, imidazolidinyl urea, iodopropynyl butylcarbamate, isothiazolinones, methenammonium chloride, methyldibromo glutaronitrile, MDM hydantoin, ortho phenylphenol, p-chloro-m-cresol, parabens (butylparaben, ethylparaben, methylparaben), phenethyl alcohol, phenoxyethanol, piroctane olamine, polyaminopropyl biguanide, polymethoxy bicyclic oxazolidine, polyoxymethylene, polyquaternium-42, potassium benzoate, potassium sorbate, propionic acid, quaternium-15, salicylic acid, selenium disulfide, sodium borate, sodium iodate, sodium hydroxymethylglycinate, sodium propionate, sodium pyrithione, sorbic acid, thimerosal, triclosan, triclocarban, undecylenic acid, zinc phenosulfonate, and zinc pyrithione.

The term “biologically active agent” as used herein, includes small molecular weight compounds, polypeptides, proteins, oligonucleotides, nucleic acids and polysaccharides.

The term “biologically active agent” thus includes, without limitation, leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N-[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, VEGF, BNP, ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormones (PTH), parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, analgesics, such as fentanyl, remifentanyl, sufentanyl, alfentanyl, lofentanyl, carfentanyl, and analogues and mixtures thereof.

The term “biologically active agent” further includes vaccines, including viruses and bacteria, protein-based vaccines, polysaccharide-based vaccine, and nucleic acid-based vaccines.

Suitable antigenic agents include, without limitation, antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linke to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diphtheriae, group A streptococcus, legionella pneumophila, neisseria meningitdis, pseudomonas aeruginosa, streptococcus pneumoniae, treponema pallidum, and vibrio cholerae, and mixtures thereof.

Additional commercially available vaccines, which contain antigenic agents, include, without limitation, flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded includes all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: β-glucan; cholera toxin B subunit; CRL1005: ABA block polymer with mean values of x=8 and y=205; gamma inulin: linear (unbranched) β-D(2→1) polyfructofuranoxyl-α-D-glucose; Gerbu adjuvant: N-acetylglucosamine-(β1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂0 (MTP); Murametide: Nac-Mur-L-Ala-D-Gln-OCH₃; Pleuran: β-glucan; QS-21; S-28463: 4-amino-a,a-dimethyl-1H-imidazo[4,5-c]quinoline-1-ethanol; sclavo peptide: VQGEESNDK∘HCl (IL-1 163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

The noted antimicrobial and biologically agents can also be in various forms, such as free bases, acids, charged or uncharged molecules, components of molecular complexes or nonirritating, pharmacologically acceptable salts.

It is to be understood that more than one antimicrobial agent and/or biologically active agent can be incorporated into the agent source, reservoirs, and/or coatings of this invention, and that the use of the term “agent formulation” in no way excludes the use of two or more such agents.

The term “microprojections”, as used herein, refers to piercing elements which are adapted to pierce or cut through the stratum corneum into the underlying epidermis layer, or epidermis and dermis layers, of the skin of a living animal, particularly, a mammal and, more particularly, a human.

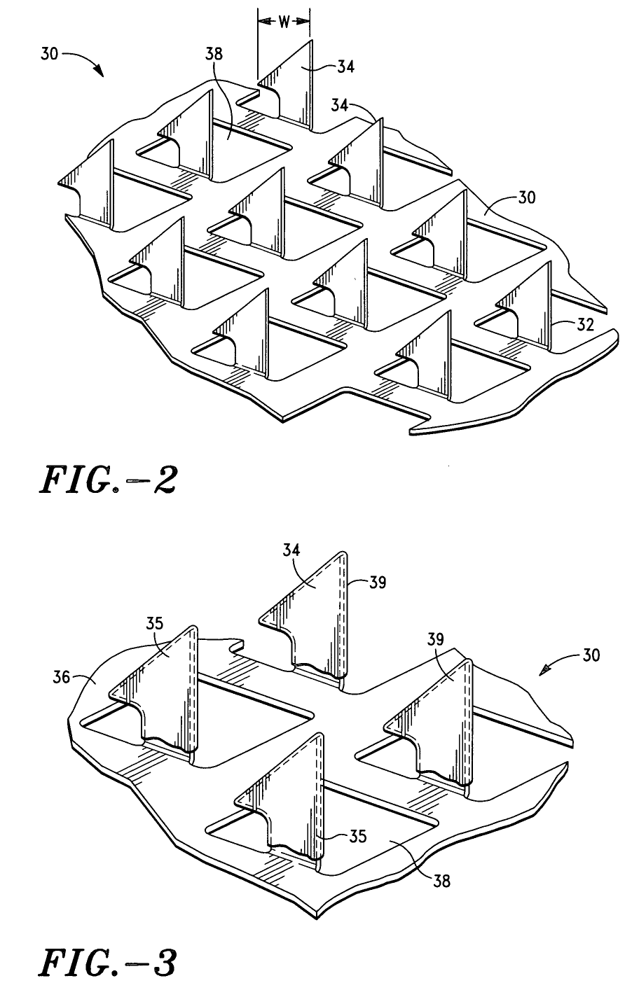

In one embodiment of the invention, the piercing elements have a projection length less than 1000 microns. In a further embodiment, the piercing elements have a projection length of less than 500 microns, more preferably, less than 250 microns. The microprojections further have a width (designated “W” in FIG. 2) in the range of approximately 25-500 microns and a thickness in the range of approximately 10-100 microns. The microprojections may be formed in different shapes, such as needles, blades, pins, punches, and combinations thereof.

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration, such as that shown in FIG. 2. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “coating formulation”, as used herein, is meant to mean and include a freely flowing composition or mixture that is employed to coat the microprojections and/or arrays thereof.

The term “biocompatible coating” and “solid coating”, as used herein, is meant to mean and include a “coating formulation” in a substantially solid state.

As indicated above, the present invention generally comprises a delivery system having a microprojection member (or system). The microprojection member includes a plurality of microprojections (or array thereof) that are adapted to pierce through the stratum corneum into the underlying epidermis layer, or epidermis and dermis layers.

Referring now to FIG. 2, there is shown one embodiment of a microprojection member 30 for use with the present invention. As illustrated in FIG. 2, the microprojection member 30 includes a microprojection array 32 having a plurality of microprojections 34. The microprojections 34 preferably extend at substantially a 90° angle from the sheet, which in the noted embodiment includes openings 38.

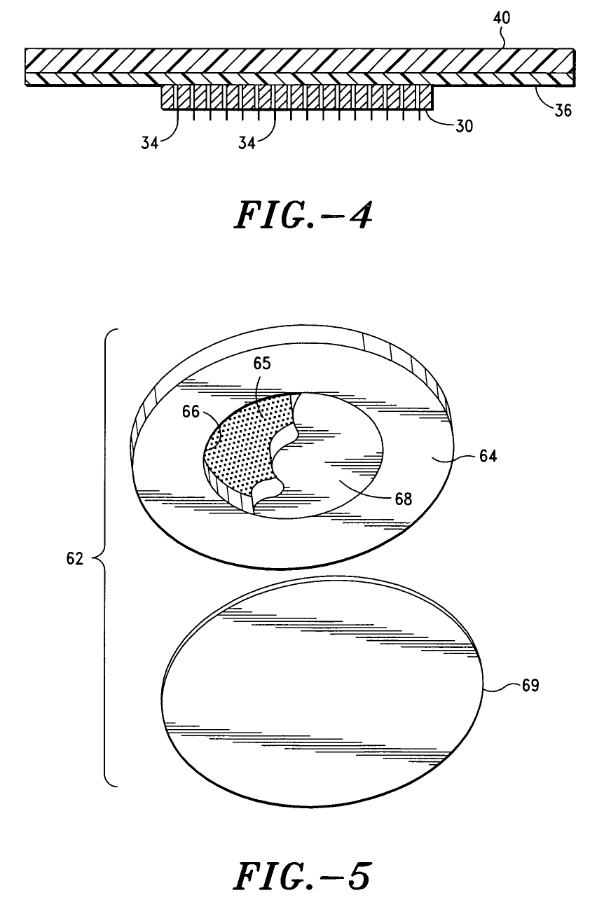

According to the invention, the sheet 36 can be incorporated into a delivery patch, including a backing 40 for the sheet 36, and can additionally include adhesive 16 for adhering the patch to the skin (see FIG. 4). In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microprojection density of at least approximately 10 microprojections/cm², more preferably, in the range of at least approximately 200-2000 microprojections/cm². Preferably, the number of openings per unit area through which the agent passes is at least approximately 10 openings/cm² and less than about 2000 openings/cm².

As indicated, the microprojections 34 preferably have a projection length less than approximately 1000 microns. In one embodiment, the microprojections 34 have a projection length of less than 500 microns, more preferably, less than 250 microns. The microprojections 34 also preferably have a width in the range of approximately 25-500 microns and thickness in the range of approximately 10-100 microns.

In a further embodiment, the microprojections 34 preferably have a length less than 145 μm, more preferably, in the range of approximately 50-145 μm, even more preferably, in the range of approximately 70-140 μm. The noted embodiments are adapted to enhance the biocompatibility of the microprojection member 30, for example, by mininimizing bleeding and irritation following application to the skin of a subject. Additionally, the microprojection member 30 exhibiting enhanced biocompatibility comprises an array preferably having a microprojection density greater than 100 microprojections/cm², more preferably, in the range of approximately 200-3000 microprojections/cm².

The microprojection member 30 can be manufactured from various metals, such as stainless steel, titanium, nickel titanium alloys, or similar biocompatible materials.

According to the invention, the microprojection member 30 can also be constructed out of a non-conductive material, such as a polymer. Alternatively, the microprojection member can be coated with a non-conductive material, such as Parylene®, or a hydrophobic material, such as Teflon®, silicon or other low energy material. The noted hydrophobic materials and associated base (e.g., photoreist) layers are set forth in U.S. Application No. 60/484,142, which is incorporated by reference herein.

Microprojection members that can be employed with the present invention include, but are not limited to, the members disclosed in U.S. Pat. Nos. 6,083,196, 6,050,988 and 6,091,975, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

As discussed in detail herein, the microprojection member (or system) of the invention includes at least one agent source or agent delivery medium (i.e., biocompatible coating, hydrogel formulation, solid film). The amount of antimicrobial agent disposed in the delivery medium will be that amount necessary to inhibit microbial growth. In practice, this will vary widely depending upon the particular antimicrobial agent, the delivery medium, the type of agent formulation, pH of the agent formulation, etc.

According to the invention, the antimicrobial agent can be contained in a biocompatible coating that is disposed on the microprojection member or in a hydrogel formulation or contained in both the biocompatible coating and hydrogel formulation.

In a further embodiment, wherein the microprojection member includes an agent-containing solid film, the antimicrobial agent can be contained in the biocompatible coating, hydrogel formulation or solid film, or in all three delivery mediums.

According to the invention, at least one biologically active agent is contained in at least one of the aforementioned delivery mediums. As will be appreciated by one having ordinary skill in the art, the present invention can also readily accommodate co-delivery of two or more biologically active agents by disposing the agents in one delivery medium or in separate delivery mediums.

In one embodiment of the invention, the microprojection member includes a biocompatible coating having at least one antimicrobial agent and at least one biologically active agent disposed therein. Upon piercing the stratum corneum layer of the skin, the agent-containing coating is dissolved by body fluid (intracellular fluids and extracellular fluids, such as interstitial fluid) and released into the skin (i.e., bolus delivery) for systemic therapy.

Referring now to FIG. 3, there is shown a microprojection member 31 having microprojections 34 that include a biocompatible coating 35. According to the invention, the coating 35 can partially or completely cover each microprojection 34. For example, the coating 35 can be in a dry pattern coating on the microprojections 34. The coating 35 can also be applied before or after the microprojections 34 are formed.

According to the invention, the coating 35 can be applied to the microprojections 34 by a variety of known methods. Preferably, the coating is only applied to those portions the microprojection member 30 or microprojections 34 that pierce the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No.2002/0132054), which is incorporated by reference herein in its entirety. As discussed in detail in the noted application, the disclosed roller coating method provides a smooth coating that is not easily dislodged from the microprojections 34 during skin piercing.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

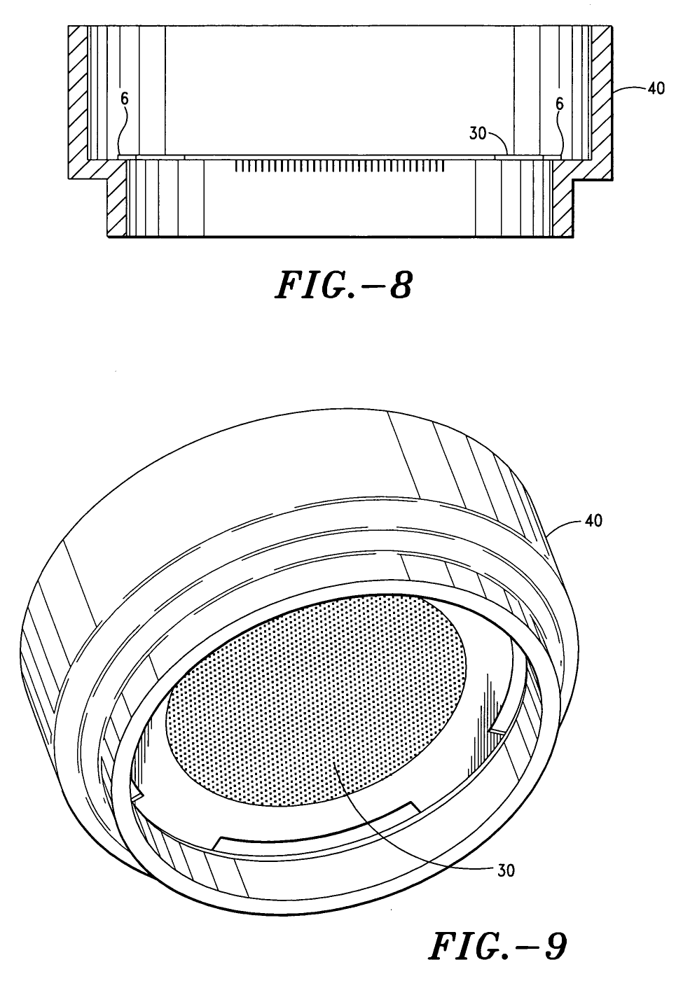

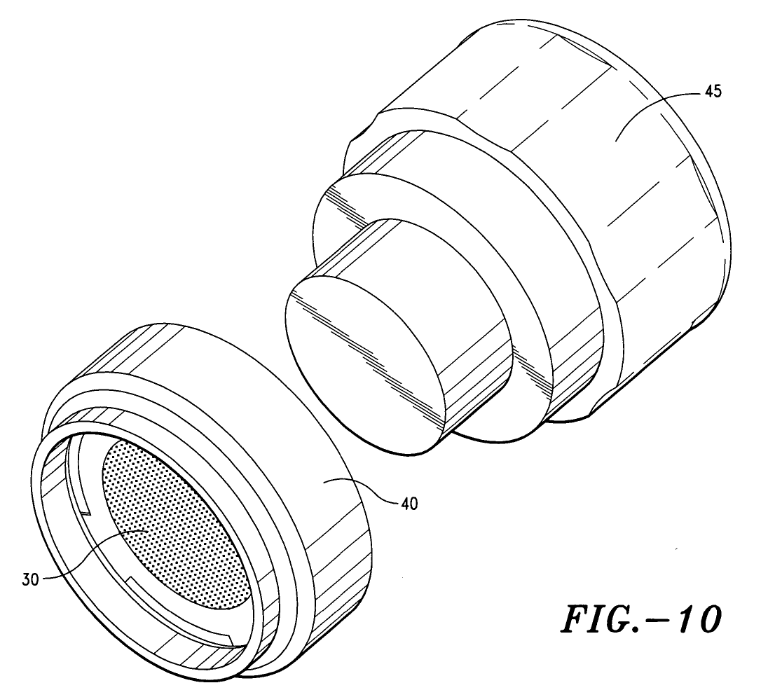

Referring now to FIGS. 8 and 9, for storage and application, the microprojection member (30 or 31) is preferably suspended in a retainer ring 40 by adhesive tabs 6, as described in detail in U.S. application Ser. No. 09/976,762 (Pub. No. 2002/0091357), which is incorporated by reference herein in its entirety.

After placement of the microprojection member in the retainer ring 40, the microprojection member is applied to the patient's skin. Preferably, the microprojection member is applied to the patient's skin using an impact applicator 45, such as shown in FIG. 10 and described in Co-Pending U.S. application Ser. No. 09/976,978, which is incorporated by reference herein in its entirety.

As indicated, according to the invention, the coating formulations applied to the microprojection member 31 to form solid biocompatible coatings can comprise aqueous and non-aqueous formulations. In one embodiment of the invention, the biocompatible coating includes at least one antimicrobial agent and at least one biologically active agent. According to the invention, the noted agents can be dissolved within a biocompatible carrier or suspended within the carrier.

Preferably, the antimicrobial agent is selected from the group consisting of 2-bromo-2-nitropropane-1,3-diol, 5-bromo-5-nitro-1,3-dioxane, 7-ethyl bicyclooxazolidine, benzalkonium chloride, benzethonium chloride, benzoic acid, benzyl alcohol, boric acid, bronopol, cetylpyridinium chloride, chlorhexidine digluconate, chloroacetamide, chlorobutanol, chloromethyl isothiazolinone and methyl isothiazoline, dimethoxane, dimethyl oxazolidine, dimethyl hydroxymethyl pyrazole, chloroxylenol, dehydroacetic acid, diazolidinyl urea, dichlorobenzyl alcohol, DMDM hydantoin, ethyl alcohol, formaldehyde, glutaraldehyde, hexachlorophene, hexetidine, hexamethy-lenetramine, imidazolidinyl urea, iodopropynyl butylcarbamate, isothiazolinones, methenammonium chloride, methyldibromo glutaronitrile, MDM hydantoin, ortho phenylphenol, p-chloro-m-cresol, parabens(butylparaben, ethylparaben, methylparaben), phenethyl alcohol, phenoxyethanol, piroctane olamine, polyaminopropyl biguanide, polymethoxy bicyclic oxazolidine, polyoxymethylene, polyquaternium-42, potassium benzoate, potassium sorbate, propionic acid, quaternium-15, salicylic acid, selenium disulfide, sodium borate, sodium iodate, sodium hydroxymethylglycinate, sodium propionate, sodium pyrithione, sorbic acid, thimerosal, triclosan, triclocarban, undecylenic acid, zinc phenosulfonate, and zinc pyrithione.

In a preferred embodiment of the invention, the biologically active agent is selected from the group consisting of small molecular weight compounds, polypeptides, proteins, oligonucleotides, nucleic acids and polysaccharides.

In one embodiment of the invention, the biologically active agent is selected from the group consisting of leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N-[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormones (PTH), parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, and mixtures thereof.

In another embodiment of the invention, the biologically active agent comprises a vaccine, including viruses and bacteria, protein-based vaccines, polysaccharide-based vaccine, and nucleic acid-based vaccines.

Suitable antigenic agents include, without limitation, antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linke to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diphtheriae, group A streptococcus, legionella pneumophila, neisseria meningitdis, pseudomonas aeruginosa, streptococcus pneumoniae, treponema pallidum, and vibrio cholerae, and mixtures thereof.

Additional commercially available vaccines, which contain antigenic agents, include, without limitation, flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded includes all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: β-glucan; cholera toxin B subunit; CRL1005: ABA block polymer with mean values of x=8 and y=205; gamma inulin: linear (unbranched) β-D(2→1)polyfructofuranoxyl-α-D-glucose; Gerbu adjuvant: N-acetylglucosamine-(β1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂0 (MTP); Murametide: Nac-Mur-L-Ala-D-Gln-OCH₃; Pleuran: β-glucan; QS-21; S-28463: 4-amino-a,a-dimethyl-1H-imidazo[4,5-c]quinoline-1-ethanol; sclavo peptide: VQGEESNDK∘HCl (IL-1β 163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used. The noted agents can be in various forms, such as free bases, acids, charged or uncharged molecules, components of molecular complexes or nonirritating, pharmaceutically acceptable salts.

Preferably, the antimicrobial agent comprises in the range of approximately 0.005-5.0 wt. % of the coating formulation.

In one embodiment of the invention, wherein ethanol is employed as a preservative, the antimicrobial agent comprises up to approximately 20 wt. % of the coating formulation. The use of ethanol and other volatiles antimicrobial agents in coating formulations is especially useful to prevent microbial growth during manufacturing.

Preferably, the biologically active agent comprises in the range of approximately 0.1-30 wt. % of the coating formulation.

In one embodiment of the invention, the coating formulation includes at least one buffer. Examples of such buffers include ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine or mixtures thereof.

In one embodiment of the invention, the coating formulation includes at least one surfactant, which can be zwitterionic, amphoteric, cationic, anionic, or nonionic. Examples of such surfactants include, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives, such as sorbitan laurate, and alkoxylated alcohols, such as laureth-4.

In one embodiment of the invention, the concentration of the surfactant is in the range of approximately 0.001-2.0 wt. % of the coating formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxy-ethylcellulose (EHEC), as well as pluronics.

In one embodiment of the invention, the concentration of the polymer presenting amphiphilic properties in the coating formulation is preferably in the range of approximately 0.01-20 wt. %, more preferably, in the range of approximately 0.03-10 wt. % of the coating formulation.

In another embodiment, the coating formulation includes a hydrophilic polymer selected from the following group: hyroxyethyl starch, dextran, poly(vinyl alcohol), poly(ethylene oxide), poly(2-hydroxyethylmethacrylate), poly(n-vinyl pyrolidone), polyethylene glycol and mixtures thereof.

In a preferred embodiment, the concentration of the hydrophilic polymer in the coating formulation is in the range of approximately 0.01-20 wt. %, more preferably, in the range of approximately 0.3-10 wt. %.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier in the coating formulation is in the range of approximately 2-70 wt. %, more preferably, in the range of approximately 5-50 wt. % of the coating formulation.

In another embodiment, the coating formulation includes a stabilizing agent, which can comprise, without limitation, a non-reducing sugar, a polysaccharide or a reducing sugar or a DNase inhibitor.

Suitable non-reducing sugars include, for example, sucrose, trehalose, stachyose, or raffinose.

Suitable polysaccharides include, for example, dextran, soluble starch, dextrin, and inulin.

Suitable reducing sugars include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

Suitable DNase inhibitors include, for example, both extracellular and intracellular DNase inhibitors. Preferred extracellular DNase inhibitors include, for example, aurintricarboxylic acid (ATA); EDTA; EGTA; and propamidine. Preferred intracellular DNases inhibitors include, for example, DMI-2, which is a polyketice metabolite of Streptomyces sp. Strain 560. In preferred embodiments of the invention, the compositions and solid coatings comprise from about 1% to about 20% by total dry weight of the DNase inhibitor.

The coating formulations and, hence, biocompatible coatings of the invention can further include a vasoconstrictor, such as those disclosed in Co-Pending U.S. application Ser. No. 10/674,626, which is incorporated by reference herein in its entirety. As set forth in the noted Co-Pending application, the vasoconstrictor is used to control bleeding during and after application on the microprojection member. Preferred vasoconstrictors include, but are not limited to, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

As will be appreciated by one having ordinary skill in the art, the addition of a vasoconstrictor to the coating formulations and, hence, solid biocompatible coatings of the invention (or the hydrogel formulations or solid film, discussed herein) is particularly useful to prevent bleeding that can occur following application of the microprojection member or array and to prolong the pharmacokinetics of the agent(s) through reduction of the blood flow at the application site and reduction of the absorption rate from the skin site into the system circulation.

The concentration of the vasoconstrictor, if employed, is preferably in the range of approximately 0.1 wt. % to 10 wt. % of the coating formulation.

In yet another embodiment ofthe invention, the coating formulation includes at least one “pathway patency modulator”, such as those disclosed in Co-Pending U.S. application Ser. No. 09/950,436, which is incorporated by reference herein in its entirety. As set forth in the noted Co-Pending application, the pathway patency modulators prevent or diminish the skin's natural healing processes thereby preventing the closure of the pathways or microslits formed in the stratum corneum by the microprojection member array. Examples of pathway patency modulators include, without limitation, osmotic agents (e.g., sodium chloride) and zwitterionic compounds (e.g., amino acids).

The term “pathway patency modulator”, as defined in the Co-Pending application, further includes anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the coating formulation includes a solubilising/complexing agent which can comprise Alpha-Cyclodextrin, Beta-Cyclodextrin, Gamma-Cyclodextrin, glucosyl-alpha-Cyclodextrin, maltosyl-alpha-Cyclodextrin, 2-hydroxypropyl-beta-Cyclodextrin, 2-hydroxypropyl-gamma-Cyclo-dextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin. Most preferred solubilising/complexing agents are beta-cyclodextrin, hydroxypropyl beta-cyclodextrin, 2-hydroxypropyl-beta-Cyclodextrin and sulfobutylether7 beta-cyclodextrin.

The concentration of the solubilising/complexing agent, if employed, is preferably in the range of approximately 1 wt. % to 20 wt. % of the coating formulation.

In another embodiment of the invention, the coating formulation includes at least one non-aqueous solvent, such as ethanol, isopropanol, methanol, propanol, butanol, pentanol, acetone, ethyl ether, benzene, amylene hydrate, methyl isobutyl ketone, propylene glycol, glycerol, and polyethylene glycols. Preferably, the solvent is present in the coating formulation in the range of approximately 5 wt. % to 99 wt. % of the coating formulation.

Other known formulation adjuvants can also be added to the coating formulations provided they do not adversely affect the necessary solubility and viscosity characteristics of the coating formulation and the physical integrity of the dried coating.

Preferably, the coating formulations have a viscosity less than approximately 500 centipoise and greater than 3 centipoise.

In one embodiment of the invention, the coating thickness is less than 25 microns, more preferably, less than 10 microns as measured from the microprojection surface.

The desired coating thickness is dependent upon several factors, including the required dosage of the biologically active agent and, hence, coating thickness necessary to deliver the dosage, the density of the microprojections per unit area of the sheet, the viscosity and concentration of the coating composition and the coating method chosen.

In all cases, after a coating has been applied, the coating formulation is dried onto the microprojections 34 by various means. In a preferred embodiment of the invention, the coated microprojection member 30 is dried in ambient room conditions. However, various temperatures and humidity levels can be used to dry the coating formulation onto the microprojections. Additionally, the coated member can be heated, lyophilized, freeze dried or similar techniques used to remove the water from the coating.

Referring now to FIG. 7, there is shown a further microprojection member (or delivery system) that can be employed within the scope of the present invention. As illustrated in FIG. 7, the member 80 includes a gel pack 62 and a microprojection assembly 70, having a microprojection member, such as the microprojection array 32.

Referring now to FIG. 5, the gel pack 62 includes a housing or ring 64 having a centrally disposed reservoir or opening 66 that is adapted to receive a predetermined amount of a hydrogel formulation 68 therein. As illustrated in FIG. 5, the ring 64 further includes a backing member 65 that is disposed on the outer planar surface of the ring 64. Preferably, the backing member 65 is impermeable to the hydrogel formulation.

In a preferred embodiment, the gel pack 62 further includes a strippable release liner 69 that is adhered to the outer surface of the gel pack ring 64 via a conventional adhesive. As described in detail below, the release liner 69 is removed prior to application of the gel pack 62 to the applied (or engaged) microprojection assembly 70.

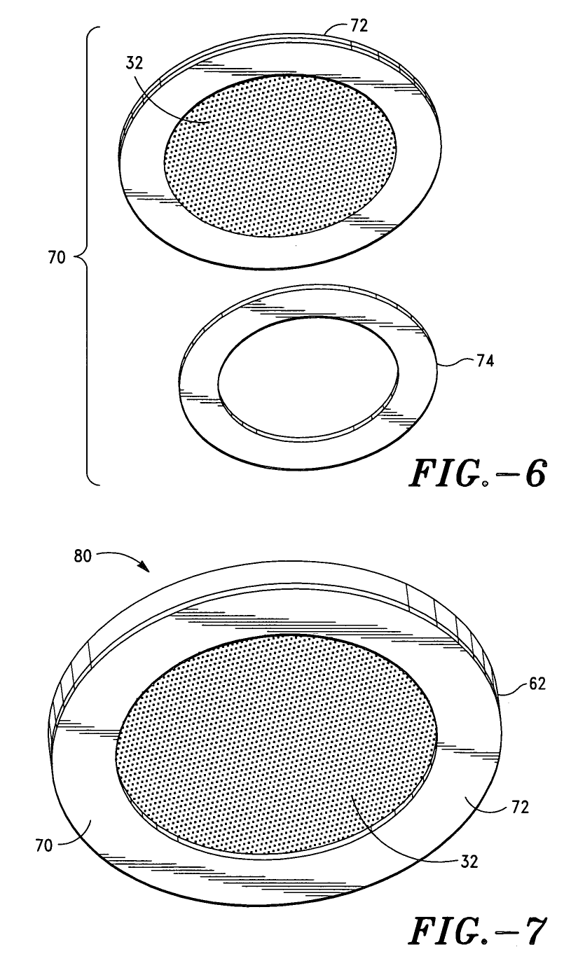

Referring now to FIG. 6, the microprojection assembly 70 further includes a backing membrane ring 72 and a skin adhesive ring 74.

Further details of the illustrated gel pack 62 and microprojection assembly 70, as well as additional embodiments thereof that can be employed within the scope of the present invention are set forth in Co-Pending Provisional Application No. 60/514,433, filed Oct. 24, 2003, which is incorporated by reference herein in its entirety.

As indicated above, in at least one embodiment of the invention, the hydrogel formulation contains at least one antimicrobial agent and at least one biologically active agent. In an alternative embodiment of the invention, the hydrogel formulation is devoid of a biologically active agent and, hence, is merely a hydration mechanism.

According to the invention, when the hydrogel formulation is devoid of a biologically active agent, the biologically active agent is either disposed in a coating on the microprojection array 32, as described above, or contained in a solid film, such as disclosed in PCT Pub. No. WO 98/28037, which is similarly incorporated by reference herein in its entirety, on the skin side of the microprojection array 32, such as disclosed in the noted Co-Pending Application No. 60/514,433 or the top surface of the array 32.

The hydrogel formulations of the invention preferably comprise aqueous formulations. In one embodiment of the invention, the hydrogel formulations include at least one antimicrobial agent and at least one biologically active agent, which can be dissolved or suspended in the hydrogel formulation.

Preferably, the antimicrobial agent comprises in the range of approximately 0.005-5 wt. % of the hydrogel formulation.

In one embodiment of the invention, wherein ethanol is employed as a preservative, the antimicrobial agent comprises up to 20 wt. % of the hydrogel formulation.

Preferably, the biologically active agent comprises in the range of approximately 0.1-30 wt. % of the hydrogel formulation.

In one embodiment of the invention, the hydrogel formulation includes at least one of the aforementioned buffers.

The hydrogel formulations of the invention preferably have sufficient surface activity to insure that the formulations exhibit adequate wetting characteristics, which are important for establishing optimum contact between the formulation and the microprojection array and skin and, optionally, the solid film.

According to the invention, adequate wetting properties are achieved by incorporating a wetting agent, such as a surfactant or polymeric material having amphiphilic properties, in the hydrogel formulation. Optionally, a wetting agent can also be incorporated in the solid film.

According to the invention, the surfactant(s) can be zwitterionic, amphoteric, cationic, anionic, or nonionic. Examples of suitable surfactants include, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laureate, and alkoxylated alcohols such as laureth-4. Most preferred surfactants include Tween 20, Tween 80, and SDS.

Examples of suitable polymers include, without limitation, cellulose derivatives, such as hydroxyethyl starch, hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics.

Preferably, the concentration of the surfactant is in the range of approximately 0.001-2 wt. % of the hydrogel formulation. The concentration of the polymer that exhibits amphiphilic properties is preferably in the range of approximately 0.5-40 wt. % of the hydrogel formulation.

As will be appreciated by one having ordinary skill in the art, the noted wetting agents can be used separately or in combinations.

In a further embodiment of the invention, the hydrogel formulation includes a solubilizing/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl-beta-Cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin. Most preferred are beta-cyclodextrin, hydroxypropyl-beta-Cyclodextrin, 2-hydroxypropyl-beta-Cyclodextrin and sulfobutylether7 beta-cyclodextrin.

In another embodiment of the invention, the hydrogel formulation includes at least one non-aqueous solvent, such as ethanol, isopropanol, acetone, propylene glycol, glycerol, and polyethylene glycols. Preferably, the solvent is present in the hydrogel formulation in the range of approximately 5 wt. % to 75 wt. % of the formulation.

According to the invention, the hydrogel formulations can similarly include at least one pathway patency modulator, such as those disclosed in Co-Pending U.S. application Ser. No. 09/950,436. As indicated above, the pathway patentcy modulator can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextran sulfate sodium, and EDTA.

The hydrogel formulation can further include at least one vasoconstrictor. Suitable vasoconstrictors include, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline, and the mixtures thereof.

The hydrogel formulations of the invention exhibit adequate viscosity so that the formulation can be contained in the gel pack, keeps its integrity during the application process, and is fluid enough so that it can flow through the microprojection assembly openings and into the skin pathways.

For hydrogel formulations that exhibit Newtonian properties, the viscosity of the hydrogel formulation is preferably in the range of approximately 2-300 Poises (P), as measured at 25° C. For shear-thinning hydrogel formulations, the viscosity, as measured at 25° C., is preferably in the range of 1.5-30 P or 0.5 and 10 P, at shear rates of 667/s and 2667/s, respectively. For dilatant formulations, the viscosity, as measured at 25° C., is preferably in the range of approximately 1.5-30 P, at a shear rate of 667/s.

In accordance with yet another embodiment of the invention, the microprojection member has top and bottom surfaces, a plurality of openings that extend through the microprojection member and a plurality of stratum corneum-piercing microprojections that project from the bottom surface of the microprojection member and includes a gel pack containing a hydrogel formulation and a solid agent-containing film. Details of the noted system are set forth in Co-Pending Application No. 60/514,433, which is incorporated by reference herein in its entirety.

Preferably, the agent-containing solid film includes at least one biologically active agent. More preferably, the agent-containing solid film includes at least one biologically active agent and at least one antimicrobial agent.

In accordance with one embodiment of the invention, the solid film is disposed proximate the top surface of the microprojection member. In another embodiment, the solid film is disposed proximate the bottom surface of the microprojection member.

In a preferred embodiment, the hydrogel formulation contains at least one antimicrobial agent is devoid of a biologically active agent.

In one embodiment, the solid film is made by casting a liquid formulation consisting of at least one antimicrobial agent, at least one biologically active agent, a polymeric material, such as hyroxyethyl starch, dextran, hydroxyethylcellulose (HEC), hydroxypropylmethylcellulose (HPMC), hydroxypropycellulose (HPC), methylcellulose (MC), hydroxyethylmethylcellulose (HEMC), ethylhydroxethylcellulose (EHEC), carboxymethylcellulose (CMC), poly(vinyl alcohol), poly(ethylene oxide), poly(2-hydroxyethymethacrylate), poly(n-vinyl pyrolidone), or pluronics, a plasticising agent, such as glycerol, propylene glycol, or polyethylene glycol, a surfactant, such as tween 20 or tween 80, and at least one volatile solvent, such as water, isopropanol, methanol, ethanol, or acetone.

In one embodiment, the liquid formulation used to produce the solid film comprises: 0.005-5 wt. % antimicrobial agent, 0.1-20 wt. % biologically active agent, 5-40 wt. % polymer, 5-40 wt. % plasticiser, 0-2 wt. % surfactant, and the balance comprising a volatile solvent.

In one embodiment of the invention, the liquid formulation used to produce the solid film includes at least one of the aforementioned buffers.

In another embodiment of the invention, the liquid formulation used to produce the solid film includes at least one of the aforementioned complexing/solubilising agents.

In a further embodiment of the invention, the liquid formulation used to produce the solid film includes at least one of the aforementioned vasoconstrictors.

In a further embodiment of the invention, the liquid formulation used to produce the solid film includes at least one of the aforementioned pathway patency modulators.

In accordance with one embodiment of the invention, the method for delivering an agent formulation of the invention includes the following steps: (i) providing a delivery system having a microprojection member 31, the microprojection member 31 including a plurality of microprojections and a biocompatible coating having at least one biologically active agent and at least one antimicrobial agent disposed therein, (ii) applying the coated microprojection member 31 to the patient's skin via an actuator, wherein the microprojections 34 pierce the skin and the agent-containing coating is dissolved by body fluid and released into the skin. The coated microprojection member 31 is preferably left on the skin for a period lasting from 5 seconds to 24 hours. Following the desired wearing time, the microprojection member 31 is removed.

In accordance with a further embodiment of the invention, the method for delivering an agent formulation of the invention includes the following steps: (i) providing a delivery system having a microprojection member 30 and a gel pack 62 including a hydrogel formulation 68 having at least one biologically active agent and at least one antimicrobial agent, (ii) applying the microprojection member 30 to the patient's skin via an actuator, wherein the microprojections pierce the stratum corneum, (iii) removing the release liner 69 from the gel pack 62 and (iv) placing the gel pack 62 on top of the applied microprojection member 30, wherein the hydrogel formulation 68 migrates into and through the microslits in the stratum corneum produced by the microprojections 34 to achieve local or systemic therapy.

The microprojection member-gel pack assembly is preferably left on the skin for a period lasting from 5 minutes to 7 days. Following the desired wearing time, the microprojection member-gel pack assembly is removed from the skin.

In a further aspect of the noted embodiment, the microprojection member 31 includes an agent-containing biocompatible coating and wherein the antimicrobial agent is present in the hydrogel formulation 68 and/or the biocompatible coating, the biologically active agent is contained in the biocompatible coating, and the hydrogel formulation 68 is devoid of a biologically active agent and, hence, is merely a hydration mechanism.

In accordance with another embodiment of the invention, the method for delivering an agent formulation of the invention includes the following steps: (i) providing a delivery system having a microprojection member 30 and a gel pack 62 including a hydrogel formulation 68 having at least one biologically active agent and at least one antimicrobial agent, (ii) applying the microprojection member 30 to the patient's skin via an actuator, wherein the microprojections 34 pierce the stratum corneum, (ii) removing the microprojection member from the patient's skin and (iii) placing the gel pack 62 on top of the pretreated skin, wherein the hydrogel formulation 68 migrates into and through the microslits in the stratum corneum produced by the microprojections 34.

The gel pack 62 is preferably left on the skin for a period lasting from 5 minutes to 7 days. Following the desired wearing time, the gel pack 62 is removed from the skin.

In a further embodiment of the invention, the method for delivering an agent formulation of the invention includes the following steps: (i) providing a delivery system having a microprojection member 30, a gel pack 62 including a hydrogel formulation having at least one biologically active agent and at least one antimicrobial agent, and a solid film having at least one biologically active agent and at least one antimicrobial agent and (ii) applying the microprojection member 30 to the patient's skin via an actuator, wherein the microprojections 34 pierce the stratum corneum, the hydrogel formulation 68 hydrates and releases the agent formulation from the solid film and the agent formulation migrates into and through the microslits in the stratum corneum produced by the microprojections 34.

The microprojection member 30 is preferably left on the skin for a period lasting from 5 seconds to 24 hours. Following the desired wearing time, the microprojection member 30 is removed from the skin.

In one aspect of the noted embodiment, the antimicrobial agent is present in the hydrogel formulation and/or the solid film, the biologically active agent is contained in the solid film, and the hydrogel formulation is devoid of a biologically active agent and, hence, is merely a hydration mechanism.

It will be appreciated by one having ordinary skill in the art that in order to facilitate drug transport across the skin barrier, the present invention can also be employed in conjunction with a wide variety of iontophoresis or electrotransport systems, as the invention is not limited in any way in this regard. Illustrative electrotransport drug delivery systems are disclosed in U.S. Pat. Nos. 5,147,296, 5,080,646, 5,169,382 and 5,169383, the disclosures of which are incorporated by reference herein in their entirety.

The term “electrotransport” refers, in general, to the passage of a beneficial agent, e.g., a drug or drug precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body may by attained in various manners.

One widely used electrotransport process, iontophoresis, involves the electrically induced transport of charged ions. Electroosmosis, another type of electrotransport process involved in the transdermal transport of uncharged or neutrally charged molecules (e.g., transdermal sampling of glucose), involves the movement of a solvent with the agent through a membrane under the influence of an electric field. Electroporation, still another type of electrotransport, involves the passage of an agent through pores formed by applying an electrical pulse, a high voltage pulse, to a membrane.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported. Additionally, other transport enhancing methods such as sonophoresis or piezoelectric devices can be used in conjunction with the invention.

When the invention is employed in conjunction with electrotransport, sonophoresis or piezoelectric systems, the microprojection assembly 70 is first applied to the skin as explained above. The release liner 69 is removed from the gel pack 62, which is part of the electrotransport, sonophoresis or piezoelectric system. This assembly is then placed on the skin template, whereby the hydrogel formulation 68 is released from the gel pack 62 and passes through the microslits in the stratum corneum formed by the microprojections 34 to achieve local or systemic therapy with additional facilitation of drug transport via the electrotransport, sonophoresis or piezoelectric processes. When the invention is employed in conjunction with one of the noted systems, the total skin contact area can be in the range of approximately 2-120 cm.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A transdermal agent delivery apparatus having a low infection potential comprising a microprojection member having a plurality of stratum corneum-piercing microprojections, at least one biologically active agent and at least one antimicrobial agent, wherein said biologically active agent and said antimicrobial agent are adapted to be delivered through microslits formed in a patient's skin by said microprojections.

2. The apparatus of claim 1, further comprising a biocompatible coating formed from a formulation of said biologically active agent and said antimicrobial agent, wherein said biocompatible coatings is disposed on said microprojections.

3. The apparatus of claim 1, further comprising a hydrogel formulation of said biologically active agent and said antimicrobial agent.

4. The apparatus of claim 1, further comprising a hydrogel formulation of said antimicrobial agent and a solid film of said biologically active agent.

5. The apparatus of claim 1, wherein said antimicrobial agent is selected from the group consisting of 2-bromo-2-nitropropane-1,3-diol, 5-bromo-5-nitro-1,3-dioxane, 7-ethyl bicyclooxazolidine, benzalkonium chloride, benzethonium chloride, benzoic acid, benzyl alcohol, boric acid, bronopol, cetylpyridinium chloride, chlorhexidine digluconate, chloroacetamide, chlorobutanol, chloromethyl isothiazolinone and methyl isothiazoline, dimethoxane, dimethyl oxazolidine, dimethyl hydroxymethyl pyrazole, chloroxylenol, dehydroacetic acid, diazolidinyl urea, dichlorobenzyl alcohol, DMDM hydantoin, ethyl alcohol, formaldehyde, glutaraldehyde, hexachlorophene, hexetidine, hexamethylenetramine, imidazolidinyl urea, iodopropynyl butylcarbamate, isothiazolinones, methenammonium chloride, methyldibromo glutaronitrile, MDM hydantoin, ortho phenylphenol, p-chloro-m-cresol, parabens (butylparaben, ethylparaben, methylparaben), phenethyl alcohol, phenoxyethanol, piroctane olamine, polyaminopropyl biguanide, polymethoxy bicyclic oxazolidine, polyoxymethylene, polyquaternium-42, potassium benzoate, potassium sorbate, propionic acid, quaternium-15, salicylic acid, selenium disulfide, sodium borate, sodium iodate, sodium hydroxymethylglycinate, sodium propionate, sodium pyrithione, sorbic acid, thimerosal, triclosan, triclocarban, undecylenic acid, zinc phenosulfonate, and zinc pyrithione.

6. The apparatus of claim 1, wherein said biologically active agent is selected from the group consisting of small molecular weight compounds, polypeptides, proteins, oligonucleotides, nucleic acids and polysaccharides.

7. The apparatus of claim 1, wherein said biologically active agent comprises an antigenic agent.

8. The apparatus of claim 2, wherein said biocompatible coating is formed from a coating formulation.

9. The apparatus of claim 8, wherein said antimicrobial agent is in the range of approximately 0.005-5.0 wt. % of said coating formulation.

10. The apparatus of claim 1, wherein said coating formulation includes at least one buffer selected from the group consisting of ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarbally acid, malonic acid, adipic acid, citraconic acid, glutaratic acid, itaconic acid, mesaconic acid, citramalic acid, dimethylopropionic acid, tiglic acid, glyceric acid, methacrylic acid, isocrotonic acid, β-hydroxybutyric acid, crotonic acid, angelic acid, hydracrylic acid, aspartic acid, glutamic acid, glycine, and mixtures thereof.

11. The apparatus of claim 1, wherein said coating formulation includes at least one surfactant selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, Tween 20, Tween 80, sorbitan derivatives, sorbitan laurate, alkoxylated alcohols, and laureth-4.

12. The apparatus of claim 1, wherein said coating formulation includes at least one polymeric material having amphiphilic properties.

13. The apparatus of claim 1, wherein said coating formulation includes a hydrophilic polymer selected from the following group consisting of hydroxyethyl starch, carboxymethyl cellulose and salts of, dextran, poly(vinyl alcohol), poly(ethylene oxide), poly(2-hydroxyethyl-methacrylate), poly(n-vinyl pyrolidone), polyethylene glycol and mixtures thereof.

14. The apparatus of claim 1, wherein said coating formulation includes a biocompatible carrier selected from the group consisting of bioengineered human albumin, polyglutamic acid, polyaspartic acid, polyhistidine, pentosan polysulfate, polyamino acids, sucrose, trehalose, melezitose, raffinose and stachyose.

15. The apparatus of claim 1, wherein said coating formulation includes a stabilizing agent selected from the group consisting of a non-reducing sugar, a polysaccharide, a reducing sugar and a Dnase inhibitor.

16. The apparatus of claim 1, wherein said coating formulation includes at least one vasoconstrictor selected from the group consisting of amidephrine, cafaminol, cyclopentaimine, deoxyepinephrine, epinephrine, felypressin, indanzoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline, and mixtures thereof.

17. The apparatus of claim 1, wherein said coating formulation includes at least one pathway patency modulator selected from the group consisting of osmotic agents, zwitterionic compounds, anti-inflammatory agents and anticoagulants.

18. The apparatus of claim 1, wherein said coating formulation includes a solubilising/complexing agent selected from the group consisting of Alpha-Cyclodextrin, Beta-Cyclodextrin, Gamma-Cyclodextrin, glucosyl-alpha-Cyclodextrin, maltosyl-alpha-Cyclodextrin, hydroxyethyl-beta-Cyclodextrin, methyl-beta-Cyclodextrin, sulfobutylether-alpha-Cyclodextrin, sulfobutylether-beta-Cyclodextrin, and sulfobutylether-gamma-Cyclodextrin.

19. The apparatus of claim 3, wherein said hydrogel formulation is in communcation with said microprojection member.

20. The apparatus of claim 19, wherein said microprojection member includes a gel pack that is adapted to receive said hydrogel formulation.

21. The apparatus of claim 19, wherein the concentration of said antimicrobial agent is in the range of approximately 0.005-5 wt. % of said hydrogel formulation.

22. The apparatus of claim 4, wherein said solid film is disposed proximate said microprojection member and said hydrogel formulation is adapted to communicate with said solid film.

23. The apparatus of claim 22, wherein said solid fim includes an antimicrobial agent.

24. The apparatus of claim 22, wherein said hydrogel formulationis devoid of said biologically active agent.

25. The apparatus of claim 22, wherein said solid film is made by casting a liquid formulation comprising said antimicrobial agent, said biologically active agent, a polymeric material, a plasticising agent, a surfactant, and at a volatile solvent.

26. The apparatus of claim 25, wherein said liquid formulation comprises 0.005-5 wt. % said antimicrobial agent, 0.1-20 wt. % said biologically active agent, 5-40 wt. % said polymeric material, 5-40 wt. % said plasticising agent, 0-2 wt. % said surfactant, and the balance comprising said volatile solvent.

27. A method of transdermally delivering a biologically active agent to a patient, comprising the steps of:
providing a delivery system including a microprojection member having a plurality of stratum corneum-piercing microprojections and a biocompatible coating disposed thereon having a biologically active agent and an antimicrobial agent; and
applying said coated microprojection member to a skin site of said patient via an actuator, whereby said plurality of stratum corneum-piercing microprojections pierce the stratum corneum and deliver said biologically active agent to said patient.

28. The method of claim 27, wherein said microprojection member remains applied to said skin site for a period of time in the range of 5 sec. to 24 hrs.

29. A method of transdermally delivering a biologically active agent to a patient, comprising the steps of:
providing a delivery system including a microprojection member having a plurality of stratum corneum-piercing microprojections and a gel pak having a hydrogel formulation of a biologically active agent and an antimicrobial agent;
applying said microprojection member to a skin site of said patient via an actuator, whereby said plurality of stratum corneum-piercing microprojections pierce the stratum corneum; and
placing said gel pak on said microprojection member, wherein said hydrogel formulation migrates into and through microslits in the stratum corneum produced by said microprojections.

30. The method of claim 29, wherein said microprojection member remains applied to said skin site for a period of time in the range of 5 min. to 7 days.

31. A method of transdermally delivering a biologically active agent to a patient, comprising the steps of:
providing a delivery system including a microprojection member having a plurality of stratum corneum-piercing microprojections and a gel pak having a hydrogel formulation of a biologically active agent and an antimicrobial agent;
applying said microprojection member to a skin site of said patient via an actuator, whereby said plurality of stratum corneum-piercing microprojections pierce the stratum corneum;
removing said microprojection member; and
placing said gel pak on said treated skin site, wherein said hydrogel formulation migrates into and through microslits in the stratum corneum produced by said microprojections.

32. The method of claim 31, wherein said microprojection member remains applied to said skin site for a period of time in the range of 5 min. to 7 days.

33. A method of transdermally delivering a biologically active agent to a patient, comprising the steps of:
providing a delivery system including a microprojection member having a plurality of stratum corneum-piercing microprojections, a solid film having a biologically active agent and an antimicrobial agent and a gel pak having a hydrogel formulation; and
applying said microprojection member to a skin site of said patient via an actuator, wherein said plurality of stratum corneum-piercing microprojections pierce the stratum corneum and wherein said hydrogel formulation hydrates and releases said biologically active agent from said solid film, allowing said biologically active agent to migrate into and through microslits in the stratum corneum produced by said microprojections.

34. The method of claim 33, wherein said microprojection member remains applied to said skin site for a period of time in the range of 5 sec. to 24 hrs.
